(19) United States
(12) Patent Application Publication
(10) Pub. No.: US 2014/0179967 A1
(43) Pub. Date: Jun. 26, 2014

(54) METHOD OF PRODUCING AROMATIC HYDROCARBONS FROM BYPRODUCTS OF AROMATIC CARBOXYLIC ACID AND/OR AROMATIC CARBOXYLIC ACID ALKYLESTER PREPARATION PROCESSES

(71) Applicants: Sang Il Lee, Daejeon (KR); Do Woan Kim, Daejeon (KR); Hyuck Jae Lee, Seoul (KR); Sang Hun Oh, Gyeonggi-do (KR); Jae Hyun Koh, Daejeon (KR); Sun Choi, Daejeon (KR); Seung Hoon Oh, Seoul (KR); Byeung Soo Lim, Daejeon (KR); Yong Seung Kim, Seoul (KR); Gyung Rok Kim, Daejeon (KR); Young Moo Park, Daejeon (KR); Jong Su Lee, Gwangju (KR)

(72) Inventors: Sang Il Lee, Daejeon (KR); Do Woan Kim, Daejeon (KR); Hyuck Jae Lee, Seoul (KR); Sang Hun Oh, Gyeonggi-do (KR); Jae Hyun Koh, Daejeon (KR); Sun Choi, Daejeon (KR); Seung Hoon Oh, Seoul (KR); Byeung Soo Lim, Daejeon (KR); Yong Seung Kim, Seoul (KR); Gyung Rok Kim, Daejeon (KR); Young Moo Park, Daejeon (KR); Jong Su Lee, Gwangju (KR)

(73) Assignees: SK GLOBAL CHEMICAL CO., LTD., Seoul (KR); SK INNOVATION CO., LTD., Seoul (KR)

(21) Appl. No.: 14/234,529

(22) PCT Filed: Jul. 25, 2012
(86) PCT No.: PCT/KR2012/005925

§ 371 Date: Feb. 14, 2014

(30) Foreign Application Priority Data
Jul. 26, 2011 (KR) 10-2011-0074082
Jul. 24, 2012 (KR) 10-2012-0080710

(51) Int. Cl. C07C 1/207

(52) U.S. Cl. CPC C07C 1/2078
USPC 585/469

(57) ABSTRACT

This invention relates to a method of producing an aromatic hydrocarbon compound from byproducts of aromatic carboxylic acid and/or aromatic carboxylic acid alkylester preparation processes using hydroprocessing under conditions of high temperature and high hydrogen pressure in the presence of a catalyst, and to a hydroprocessing catalyst used therein.

### TECHNICAL FIELD

The present invention relates to a method of producing aromatic hydrocarbons (e.g. benzene, toluene, xylene, aromatic hydrocarbons having 9 or more carbons (hereinafter, referred to as C9+ aromatic hydrocarbons), etc.) from byproducts of aromatic carboxylic acid and/or aromatic carboxylic acid alkylester preparation processes using hydroprocessing under conditions of high temperature and high hydrogen pressure in the presence of a catalyst.

### BACKGROUND ART

Aromatic acids are used to manufacture synthetic fibers, synthetic resins, plasticizers or fine chemicals. Although partial oxidation is applied to produce terephthalic acid from p-xylene, isophthalic acid from m-xylene, phthalic acid or its anhydrides from o-xylene, and benzoic acid from toluene, it also produces a variety of solid byproducts including side-products, in addition to aromatic acids, and such byproducts have no appropriate use and are mainly disposed of. The byproducts of such partial oxidation include unreacted reactants, reaction intermediates or small amounts of products, such as tolualdehyde (TAD), benzoic acid (BZA), toluic acid (TA), carboxy benzaldehyde (CBA), phthalic acid (PA), isophthalic acid (IPA), terephthalic acid (TPA), etc.

Aromatic carboxylic acid alkylesters are prepared by subjecting aromatic carboxylic acids to esterification with alcohol. Concretely, the reaction with methanol enables terephthalic acid to be changed into dimethyl terephthalate (DMT), isophthalic acid to be changed into dimethyl isophthalate (DMIP), phthalic acid to be changed into dimethyl phthalate (DMP), and benzoic acid to be changed into methyl benzoate (MBZ).

In the preparation of aromatic carboxylic acid alkylesters, impurities including reaction intermediates or side-products are produced, and purification is essential to remove the impurities. In this case, a large amount of byproducts are generated. The byproducts of such esterification include unreacted reactants, side-products, reaction intermediates or small amounts of products, such as xylene (Xyl), methyl benzoate (MBZ), methyl toluate (MT), phenol, methyl-formylbenzoate (MFB), DMT, DMIP, DMP, etc. Such reaction byproducts have no appropriate use and are mainly discarded.

### DISCLOSURE OF INVENTION

### Technical Problem

Accordingly, the present invention has been made keeping in mind the above problems occurring in the related art, and an object of the present invention is to provide the production of aromatic hydrocarbons (e.g. benzene, toluene, xylene, C9+ aromatic hydrocarbons, etc.) by subjecting byproducts of aromatic carboxylic acid and/or aromatic carboxylic acid alkylester preparation processes to hydroprocessing under conditions of high temperature and high hydrogen pressure in the presence of a catalyst.

### Solution to Problem

In the present specification, byproducts include unreacted reactants, side-products, reactants, and small amounts of products generated in aromatic carboxylic acid and/or aromatic carboxylic acid alkylester preparation processes. Also, most of them are discarded as waste and thus the byproducts may be considered to include waste.

The present invention provides a method of producing aromatic hydrocarbons from byproducts of aromatic carboxylic acid and/or aromatic carboxylic acid alkylester preparation processes using hydrodeoxygenation, decarboxylation, or hydrocracking under conditions of high temperature and high hydrogen pressure in the presence of a hydroprocessing catalyst, and also provides a hydrogenation catalyst used therein. This process may be performed in batch or continuous mode, and the present invention will be more fully described as below.

According to an embodiment of the present invention, the method may include a) preparing byproducts of aromatic carboxylic acid and/or aromatic carboxylic acid alkylester preparation processes, and b) converting the byproducts into aromatic hydrocarbons in the presence of a hydroprocessing catalyst, thus obtaining a product including aromatic hydrocarbons.

According to an embodiment of the present invention, the method may further include c) separating the product obtained in b) into liquid aromatic hydrocarbons, gas components and water.

According to an embodiment of the present invention, the method may further include d) separating the liquid aromatic hydrocarbons obtained in c) into benzene, toluene, xylene, and C9+ aromatic hydrocarbon compounds.

According to an embodiment of the present invention, the method may further include e) recycling a part of the liquid aromatic hydrocarbons, the gas components, or the hydrogen of the gas components from which impurities have been removed, as obtained in c), into a) so as to be mixed with the byproducts. According to an embodiment of the present invention, the method may further include f) recycling a part of the aromatic hydrocarbons obtained in d) into a) so as to be mixed with the byproducts.

According to an embodiment of the present invention, the byproducts of aromatic carboxylic acid and/or aromatic carboxylic acid alkylester preparation processes may include aromatic compounds in which a benzene ring has a substituent that contain hydrocarbons or oxygen, and the substituent may be an alkyl group, an aldehyde group, a carboxyl group, an ester group, a hydroxyl group, or an alkoxy group.

According to an embodiment of the present invention, the aromatic carboxylic acid may be terephthalic acid, isophthalic acid, phthalic acid and anhydrides thereof, or benzoic acid, and the aromatic carboxylic acid alkylester may be dimethyl terephthalate, dimethyl isophthalate or dimethyl phthalate. Furthermore, the aromatic carboxylic acid may be terephthalic acid, and the aromatic carboxylic acid alkylester may be dimethyl terephthalate.

According to an embodiment of the present invention, upon preparation of aromatic carboxylic acid and/or aromatic carboxylic acid alkylester, unreacted reactants, side-products reaction intermediates or small amounts of products, such as tolualdehyde (TAD), benzoic acid (BZA), toluic acid (TA), carboxy benzaldehyde (CBA), phthalic acid (PA), isophthalic acid (IPA), terephthalic acid (TPA), xylene, (Xyl), methyl benzoate (MBZ), methyl toluate (MT), phenol, methyl-formylbenzoate (MFB), dimethyl terephthalate (DMT), dimethyl isophthalate (DMIP) or dimethyl phthalate (DP) may be present, and are typically represented by Chemical Formula 1 below.

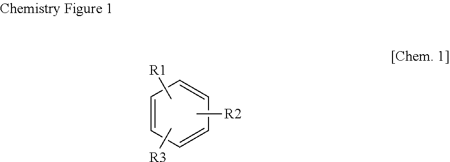

(where R₁, R₂ or R₃ is H, CH₃, CHO, COON, COOCH₃, OH, OR or CH₂OH)

According to an embodiment of the present invention, the byproducts of aromatic carboxylic acid and/or aromatic carboxylic acid alkylester preparation processes may be reacted under conditions of high temperature and high hydrogen pressure in the presence of a hydroprocessing catalyst, thus producing aromatic hydrocarbons such as benzene, toluene, xylene, C9+ aromatic hydrocarbons, etc., and hydrogen. The typical reaction scheme thereof is represented as below.

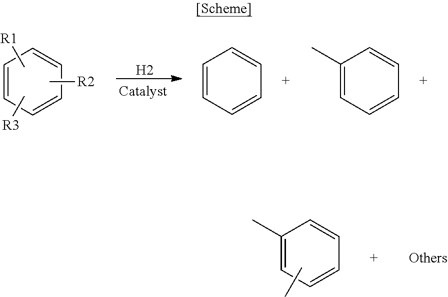

With reference to FIGS. 1 and 2, exemplary embodiments of the invention are described.

In an embodiment of the present invention, byproducts of aromatic carboxylic acid and/or aromatic carboxylic acid alkylester preparation processes are converted into aromatic hydrocarbons using hydrodeoxygenation, decarboxylation or hydrocracking in the presence of a hydroprocessing catalyst. The resulting products may be separated into liquid aromatic hydrocarbons, gas components and water, using gas/liquid and oil/water separation. The gas components are separated using gas/liquid separation, and water and liquid aromatic hydrocarbons are separated using oil/water separation. Only the byproducts may be added into a hydroprocessing reactor. In some cases, in order to facilitate the addition of byproducts and alleviate reactive heat generated upon hydroprocessing, a part of the produced liquid aromatic hydrocarbons or the unreacted reactant may be recycled to the inlet of the reactor so as to be mixed with the byproducts. Furthermore, paraffin, naphthene or aromatic hydrocarbon compounds, such as ethylbenzene, hexadecane, cyclohexane, etc., may be used as a reaction solvent, in addition to the produced liquid aromatic hydrocarbons. The separated gas components include a large amount of hydrogen and may thus be recycled to the inlet of the reactor, as necessary. In this case, impurities are removed from the gas components to ensure a favorable hydroprocessing reaction, and hydrogen may be recycled to the inlet of the reactor so as to be mixed with byproducts.

The separated liquid aromatic hydrocarbons may be further separated into benzene, toluene, xylene, and C9+ aromatic hydrocarbons. To this end, distillation or the like may be applied, and a variety of separation methods known in the art may be used. Also, a part of the aromatic hydrocarbons separated from the liquid aromatic hydrocarbons may be recycled to the inlet of the reactor so as to be mixed with the byproducts.

According to an embodiment of the present invention, hydroprocessing may be performed under conditions of a reaction temperature of 150˜500° C. and a reaction pressure of 1˜300 kgf/cm².

According to an embodiment of the present invention, the hydroprocessing catalyst may be used without being supported, or may be provided in the form of a supported catalyst by supporting a metal component on a support such as inorganic oxide or carbon. The inorganic oxide may be one or more selected from among silica, alumina, silica-alumina, zirconia, titania, aluminum phosphate, niobia, clay and zeolite.

According to an embodiment of the present invention, the catalyst used in the hydroprocessing reaction may include one or more metals selected from the group consisting of metals of Groups 6, 7, 8, 9, 10, and 11 among a total of 18 groups on the periodic table. The metal component may be one or more selected from the group consisting of Cr, Mo, W, Ru, Co, Rh, Ni, Pd, Pt, Cu, and Fe. Also, the metal may be one or more selected from the group consisting of Mo, W, Fe, Ru, Co, Ni, and Cu. Also, in order to maintain the reaction activity, the hydroprocessing catalyst may be provided in the form of the above metal or sulfide, phosphide or oxide thereof.

### Advantageous Effects of Invention

According to the present invention, byproducts of aromatic carboxylic acid and/or aromatic carboxylic acid alkylester preparation processes, which are currently disposed of, can be converted into aromatic hydrocarbons which can be utilized in a variety of end uses. Also, this invention is useful in terms of recycling resources and solving environmental pollution problems.

### BRIEF DESCRIPTION OF DRAWINGS

FIG. 1 schematically illustrates hydroprocessing of byproducts, and separation of the produced aromatic hydrocarbons, gas components including hydrogen, and water to produce an aromatic hydrocarbon mixture; and

FIG. 2 schematically illustrates additional separation of the aromatic hydrocarbon mixture of Stream 4 of FIG. 1 into benzene, toluene, xylene and C9+ aromatic hydrocarbons using a hydrocarbon separation unit, or recycle of a part of the product into a reactor.

### BEST MODE FOR CARRYING OUT THE INVENTION

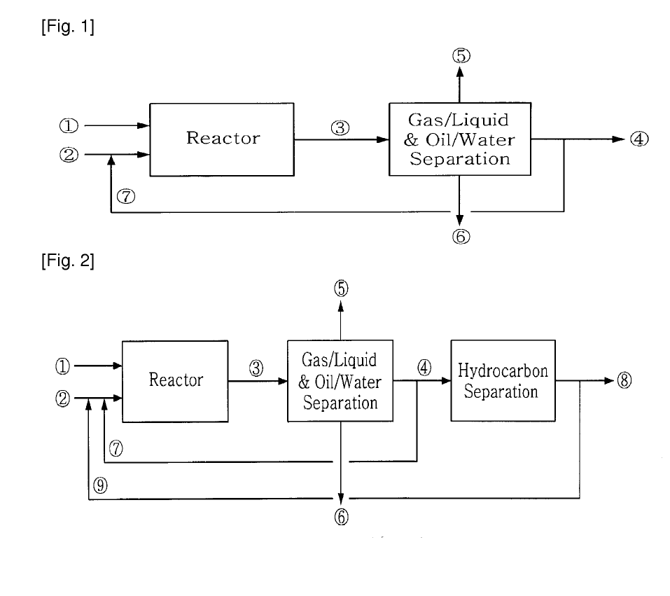

FIGS. 1 and 2 are specified below.

As illustrated in FIG. 1, hydrogen as Stream 1 and byproducts alone or in mixtures with a solvent as Stream 2 are fed into a reactor, and in order to maintain reaction activity, a sulfur compound may also be fed in depending on the type of catalyst. The mixture resulting from the hydroprocessing reaction is composed of oxygen-free aromatic hydrocarbons, gas components including hydrogen, and water. Such a mixture (Stream 3) is fed into a gas/liquid and oil/water separation unit, thus obtaining gas components as Stream 5, water as Stream 6 which will be removed, and an aromatic hydrocarbon product composed mainly of benzene, toluene and xylene as Stream 4. In order to facilitate the addition of byproducts and alleviate the reactive heat generated in the hydroprocessing reaction, a part of the produced aromatic hydrocarbons may be recycled via Stream 7. Also, the gas components of Stream 5 include a large amount of hydrogen and may thus be fed again to Stream 1. In this case, hydrogen may be fed after impurities have been removed therefrom, in order to favorably carry out the hydroprocessing reaction. As illustrated in FIG. 2, the aromatic hydrocarbon mixture of Stream 4 of FIG. 1 is fed to a hydrocarbon separation unit and thus may be separated into benzene, toluene, xylene and C9+ aromatic hydrocarbons (Stream 8) using distillation or the like. A part of the product of Stream 4 or 8 may be recycled to the reactor via Stream 7 or 9, as necessary.

Below, the present invention will be more fully described through the following examples and is not limited to the examples.

### EXAMPLE 1

20 wt % of byproducts of aromatic carboxylic acid preparation processes composed mainly of BZA (21.4%), TAD (41.4%), CBA (8.3%) and IPA (23.2%) was mixed with ethylbenzene, after which 0.1 wt % of dimethyldisulfide was added thereto, thus preparing a reactant, which was then fed at a rate of 0.5 cc/min into a fixed-bed continuous reactor filled with 5 cc of a NiMoS/ZrO₂ catalyst. As such, the reaction was carried out under conditions of a reaction temperature of 300° C., a hydrogen pressure of 30 atm, and H₂=50 cc/min, producing aromatic hydrocarbon compounds composed mainly of benzene, toluene and xylene.

Preparation of NiMo/ZrO₂ Catalyst

This catalyst was prepared by supporting about 10 wt % of Mo and about 3 wt % of Ni on a zirconia support having a diameter of 1 mm. Specifically, the zirconia support was impregnated with an aqueous solution of ammonium heptamolybdate tetrahydrate (AHM) dissolved in distilled water, dried at 150° C. for 2 hr, and then continuously fired at 500° C. for 2 hr, thus preparing MoO₃/ZrO₂. Thereafter, the MoO₃/ZrO₂ catalyst was impregnated with a solution of nickel nitrate hexahydrate (NNH) in distilled water, dried at 150° C. for 2 hr, and then continuously fired at 500° C. for 2 hr, thus preparing a NiMo/ZrO₂ catalyst.

Examples of the Mo precursor include not only AHM but also molybdenum acetate, molybdenum chloride, molybdenum hexacarbonyl, phosphomolybdic acid, molybdic acid, etc., and also, the Ni precursor may be provided in various forms, and the present invention is not limited thereto.

Sulfidation of Catalyst

5 cc of the catalyst prepared as above was charged into a fixed-bed continuous reactor, heated to 320° C. while allowing H₂ to flow at a rate of 16 cc/min and 5 wt % dimethyldisulfide-added hexadecane to flow at a rate of 0.1 cc/min at room temperature and at a reaction pressure of 45 atm, and then pretreated at 350° C. for 3 hr.

A variety of sulfur compounds may be used in sulfiding the catalyst, and the present invention does not limit the sulfiding agent to dimethyldisulfide. Also, hexadecane is used as a solvent which mixes the sulfur compound, but the present invention is not limited thereto.

### EXAMPLE 2

20 wt % of byproducts of aromatic carboxylic acid preparation processes composed mainly of BZA (21.4%), TAD (41.4%), CBA (8.3%) and IPA (23.2%) was mixed with ethylbenzene, after which 0.1 wt % of dimethyldisulfide was added thereto, thus preparing a reactant, which was then fed at a rate of 0.5 cc/min into a fixed-bed continuous reactor filled with 5 cc of a CoMoS/TiO₂ catalyst. As such, the reaction was carried out under conditions of a reaction temperature of 300° C., a hydrogen pressure of 30 atm and H₂=50 cc/min, producing aromatic hydrocarbon compounds composed mainly of benzene, toluene and xylene.

Preparation and Sulfidation of CoMo/TiO₂ Catalyst

This catalyst was prepared by supporting about 10 wt % of Mo and about 3 wt % of Co on a titania support having a diameter of 1 mm. Specifically, the titania support was impregnated with an aqueous solution of AHM dissolved in distilled water, dried at 150° C. for 2 hr, and then continuously fired at 500° C. for 2 hr, thus preparing MoO₃/TiO₂.

Thereafter, the MoO₃/TiO₂ catalyst was impregnated with a solution of cobalt nitrate hexahydrate (CNH) in distilled water, dried at 150° C. for 2 hr, and then continuously fired at 500° C. for 2 hr, thus preparing a CoMo/TiO₂ catalyst. The catalyst was sulfided under the same conditions as in Example 1, and the reaction test was performed.

### EXAMPLE 3

20 wt % of a mixture of byproducts of aromatic carboxylic acid and/or aromatic carboxylic acid alkylester preparation processes composed mainly of MBZ (19.5%), TAD (37.7%), CBA (7.5%), IPA (21.1%), MBZ (1.1%), MT (1.7%), MFB (5.0%) and DMT (0.9%) was dissolved in ethylbenzene, after which 0.1 wt % of dimethyldisulfide was added thereto, thus preparing a reactant, which was then fed at a rate of 0.5 cc/min into a fixed-bed continuous reactor filled with 5 cc of a NiMoP/Al₂O₃ catalyst. As such, the reaction was carried out under conditions of a reaction temperature of 280° C., a reaction pressure of 50 atm and H₂=50 cc/min, producing aromatic hydrocarbon compounds composed mainly of benzene, toluene and xylene.

Preparation and Sulfidation of NiMoP/Al₂O₃ Catalyst

Alumina having a diameter of 1 mm was impregnated with an aqueous H₃PO₄ solution, and fired at 500° C. for 2 hr, followed by performing the same procedures as in Example 1, thus preparing a catalyst comprising about 15 wt % of Mo, about 4 wt % of Ni and about 3 wt % of P, which was then sulfided. In the case of P, a variety of precursors may be used.

### EXAMPLE 4

20 wt % of byproducts of aromatic carboxylic acid alkylester preparation processes composed mainly of MBZ (1.3%), MT (10.0%), MFB (73.0%) and DMT (12.8%) was dissolved in ethylbenzene, after which 0.1 wt % of dimethyldisulfide was added thereto, thus preparing a reactant, which was then fed at a rate of 0.5 cc/min into a fixed-bed continuous reactor filled with 5 cc of a CoMoS/AlPO₄ catalyst. As such, the reaction was carried out under conditions of a reaction temperature of 300° C., a reaction pressure of 30 atm and H₂=50 cc/min, producing aromatic hydrocarbon compounds composed mainly of benzene, toluene, and xylene.

Preparation and Sulfidation of CoMo/AlPO₄ Catalyst

This catalyst was prepared and sulfided in the same manner as in Example 2, with the exception that aluminum phosphate having a diameter of 1 mm was used as a support.

### EXAMPLE 5

20 wt % of byproducts of aromatic carboxylic acid alkylester preparation processes composed mainly of MBZ (11.9%), MT (18.7%), MFB (54.8%) and DMT (9.6%) was dissolved in ethylbenzene, after which 0.1 wt % of dimethyldisulfide was added thereto, thus preparing a reactant, which was then fed at a rate of 0.5 cc/min into a fixed-bed continuous reactor filled with 5 cc of a NiWS/Nb₂O₅ catalyst. As such, the reaction was carried out under conditions of a reaction temperature of 300° C., a reaction pressure of 30 atm and H₂=50 cc/min, producing aromatic hydrocarbon compounds composed mainly of benzene, toluene and xylene.

Preparation and Sulfidation of NiWS/Nb₂O₅ Catalyst

This catalyst was prepared by supporting about 15 wt % of W and about 3 wt % of Ni on a niobia support having a diameter of 1 mm. Specifically, the niobia support was impregnated with an aqueous solution of ammonium metatungstate hydrate (AMT) dissolved in distilled water, dried at 150° C. for 2 hr and then continuously fired at 500° C. for 2 hr, thus preparing WO₃/Nb₂O₅.

The WO₃/Nb₂O₅ catalyst was impregnated with a solution of NNH in distilled water, dried at 150° C. for 2 hr and continuously fired at 500° C. for 2 hr, thus preparing a NiW/Nb₂O₅ catalyst, which was then sulfided in the same manner as in Example 1.

### EXAMPLE 6

20 wt % of a mixture of byproducts of aromatic carboxylic acid and/or aromatic carboxylic acid alkylester preparation processes composed mainly of MBZ (19.5%), TAD (37.7%), CBA (7.5%), IPA (21.1%), MBZ (1.1%), MT (1.7%), MFB (5.0%) and DMT (0.9%) was mixed with ethylbenzene, after which 0.1 wt % of dimethyldisulfide was added thereto, thus preparing a reactant, which was then fed at a rate of 0.5 cc/min into a fixed-bed continuous reactor filled with 5 cc of a CuMoS/Al₂O₃ catalyst. As such, the reaction was carried out under conditions of a reaction temperature of 330° C., a hydrogen pressure of 30 atm and H₂=50 cc/min, producing aromatic hydrocarbon compounds composed mainly of benzene, toluene, and xylene.

Preparation and Sulfidation of CuMo/Al₂O₃ Catalyst

This catalyst was prepared by supporting about 10 wt % of Mo and about 3 wt % of Co on an alumina support having a diameter of 1 mm. Specifically, the alumina support was impregnated with an aqueous solution of AHM dissolved in distilled water, dried at 150° C. for 2 hr and then continuously fired at 500° C. for 2 hr, thus preparing MoO₃/Al₂O₃.

The MoO₃/Al₂O₃ catalyst was impregnated with a solution of copper nitrate hydrate in distilled water, dried at 150° C. for 2 hr and continuously fired at 500° C. for 2 hr, thus preparing a CuMo/Al₂O₃ catalyst, which was then sulfided under the same conditions as in Example 1, and the reaction test was performed.

### EXAMPLE 7

A mixture of byproducts of aromatic carboxylic acid and/or aromatic carboxylic acid alkylester preparation processes composed mainly of MBZ (8.7%), TAD (15.0%), CBA (4.1), IPA (11.6%), MBZ (21.5%), MT (23.0%), MFB (10.0%) and DMT (5.0%) was added with 0.5 wt % of dimethyldisulfide, thus preparing 10 g of a reactant, which was then fed together with 50 g of hexadecane, 1.5 g of NiMoS/SiO₂ catalyst having a size of 100 mesh or less and hydrogen into a batch reactor. As such, the reaction was carried out under conditions of a reaction temperature of 320° C., a reaction pressure of 55 atm and a reaction time of 6 hr, producing aromatic hydrocarbon compounds composed mainly of benzene, toluene and xylene.

Preparation and Pretreatment of NiMo/SiO₂ Catalyst

This catalyst was prepared and sulfided in the same manner as in Example 1, with the exception that silica was used instead of zirconia. The resulting catalyst was milled to a size of 100 mesh or less.

The compositions of the liquid reaction products of Examples 1 to 7 and the amounts (unit: %) thereof are given in Table 1 below (ethylbenzene used as the mixing solvent is not shown).

TABLE 1

| | Benzene | Toluene | Xylene | C9+ Aromatic | Others |
|---|---|---|---|---|---|
| Ex. 1 | 5.1 | 36.1 | 49.7 | 7.8 | 1.3 |
| Ex. 2 | 3.9 | 36.7 | 51.2 | 6.6 | 1.6 |
| Ex. 3 | 4.8 | 35.7 | 50.7 | 7.4 | 1.4 |
| Ex. 4 | 1.4 | 30.4 | 64.4 | 2.3 | 1.5 |
| Ex. 5 | 2.3 | 31.8 | 60.7 | 3.7 | 1.5 |
| Ex. 6 | 5.2 | 36.9 | 49.3 | 6.9 | 1.7 |
| Ex. 7 | 23.5 | 48.0 | 27.5 | 0.2 | 0.8 |

### EXAMPLE 8

20 wt % of byproducts of aromatic carboxylic acid preparation processes composed mainly of BZA (21.4%), TAD (41.4%), CBA (8.3%) and IPA (23.2%) was mixed with hexadecane, after which 0.1 wt % of dimethyldisulfide was added thereto, thus preparing a reactant, which was then fed at a rate of 0.3 cc/min into a fixed-bed continuous reactor filled with 3 cc of a Ni₂P/TiO₂catalyst. As such, the reaction was carried out under conditions of a reaction temperature of 330° C., a reaction pressure of 30 atm and H₂=50 cc/min, producing aromatic hydrocarbon compounds composed mainly of benzene, toluene and xylene.

Preparation of Ni₂P/TiO₂ Catalyst

This catalyst was prepared by supporting about 6 wt % of Ni and about 4 wt % of P on a titania support having a diameter of 1 mm. Specifically, the titania support was impregnated with an aqueous solution of NNH and ammonium phosphate (AP) in distilled water, dried at 150° C. for 2 hr, and continuously fired at 500° C. for 2 hr, thus preparing Ni₂P/TiO₂. The present invention does not limit the Ni and P metal precursors to the above precursors.

Pretreatment of Catalyst

5 cc of the catalyst prepared as above was charged into a cylindrical reactor, heated to 600° C. while allowing H₂ to flow at a rate of 200 cc/min at room temperature and at a reaction pressure of 30 atm, and then pretreated at 600° C. for 2 hr.

### EXAMPLE 9

20 wt % of a mixture of byproducts of aromatic carboxylic acid and/or aromatic carboxylic acid alkylester preparation processes composed mainly of MBZ (19.5%), TAD (37.7%), CBA (7.5%), IPA (21.1%), MBZ (1.1%), MT (1.7%), MFB (5.0%) and DMT (0.9%) was mixed with hexadecane, after which 0.1 wt % of dimethyldisulfide was added thereto, thus preparing a reactant, which was then fed at a rate of 0.3 cc/min into a fixed-bed continuous reactor filled with 5 cc of a Co₂P/SiO₂.Al₂O₃ catalyst. As such, the reaction was carried out under conditions of a reaction temperature of 350° C., a reaction pressure of 25 atm and H₂=30 cc/min, producing aromatic hydrocarbon compounds composed mainly of benzene, toluene, and xylene.

Preparation and Pretreatment of Co₂P/Si₂.Al₂O₃ Catalyst

This catalyst was prepared by supporting about 6 wt % of Co and about 4 wt % of P on a silica-alumina support having a diameter of 1 mm. Specifically, the silica-alumina support was impregnated with an aqueous solution of CNH and AP in distilled water, dried at 150° C. for 2 hr, and continuously fired at 500° C. for 2 hr, thus preparing Coₑ P/SiO₂Al₂O₃. The prepared catalyst was pretreated and reacted under the same conditions as in Example 8, with the exception that the pretreatment temperature was 700° C. The present invention does not limit the Co and P metal precursors to the above precursors.

### EXAMPLE 10

20 wt % of byproducts of aromatic carboxylic acid alkylester preparation processes composed mainly of MBZ (11.9%), MT (18.7%), MFB (54.8%) and DMT (9.6%) was mixed with hexadecane, after which 0.1 wt % of dimethyldisulfide was added thereto, thus preparing a reactant, which was then fed at a rate of 0.2 cc/min into a fixed-bed continuous reactor filled with 5 cc of a Fe₂P/TiO₂ catalyst. As such, the reaction was carried out under conditions of a reaction temperature of 350° C., a reaction pressure of 40 atm and H₂=20 cc/min, producing aromatic hydrocarbon compounds composed mainly of benzene, toluene, and xylene.

Preparation and Pretreatment of Fe₂P/TiO₂ Catalyst

This catalyst was prepared by supporting about 7 wt % of Fe and about 4 wt % of P on a titania support having a diameter of 1 mm. Specifically, the titania support was impregnated with an aqueous solution of iron (III) nitrate and AP in distilled water, dried at 150° C. for 2 hr, and continuously fired at 500° C. for 2 hr, thus preparing Fe₂P/TiO₂. The prepared catalyst was pretreated and reacted under the same conditions as in Example 9. The present invention does not limit the Fe and P precursors to the above precursors.

### EXAMPLE 11

20 wt % of byproducts of aromatic carboxylic acid alkylester preparation processes composed mainly of MBZ (11.9%), MT (18.7%), MFB (54.8%) and DMT (9.6%) was mixed with hexadecane, after which 0.1 wt % of dimethyldisulfide was added thereto, thus preparing a reactant, which was then fed at a rate of 0.2 cc/min into a fixed-bed continuous reactor filled with 5 cc of a WP/SiO₂ catalyst. As such, the reaction was carried out under conditions of a reaction temperature of 350° C., a reaction pressure of 30 atm and H₂=20 cc/min, producing aromatic hydrocarbon compounds composed mainly of benzene, toluene and xylene.

Preparation of WP/SiO₂ Catalyst

This catalyst was prepared by supporting about 8 wt % of W and about 3 wt % of P on a silica support having a diameter of 1 mm. Specifically, the silica support was impregnated with an aqueous solution of AMT and AP in distilled water, dried at 150° C. for 2 hr, and continuously fired at 500° C. for 2 hr, thus preparing WP/SiO₂. The prepared catalyst was pretreated and reacted under the same conditions as in Example 8. The present invention does not limit the W and P precursors to the above precursors.

### EXAMPLE 12

20 wt % of byproducts of aromatic carboxylic acid preparation processes composed mainly of BZA (21.4%), TAD (41.4%), CBA (8.3%) and IPA (23.2%) was mixed with hexadecane, after which 0.1 wt % of dimethyldisulfide was added thereto, thus preparing a reactant, which was then fed at a rate of 0.3 cc/min into a fixed-bed continuous reactor filled with 5 cc of a MoP/ZrO₂ catalyst. As such, the reaction was carried out under conditions of a reaction temperature of 340° C., a reaction pressure of 30 atm and H₂=30 cc/min, producing aromatic hydrocarbon compounds composed mainly of benzene, toluene, and xylene.

Preparation of MoP/ZrO₂ Catalyst

This catalyst was prepared by supporting about 7 wt % of Mo and about 4 wt % of P on a zirconia support having a diameter of 1 mm. Specifically, the zirconia support was impregnated with an aqueous solution of AP and AHM in distilled water, dried at 150° C. for 2 hr, and continuously fired at 500° C. for 2 hr, thus preparing MoP/ZrO₂. The prepared catalyst was pretreated and reacted under the same conditions as in Example 8. The present invention does not limit the Mo and P precursors to the above precursors.

The compositions of the liquid reaction products of Examples 8 to 12 and the amounts (unit: %) thereof are given in Table 2 below (hexadecane used as the mixing solvent is not shown).

TABLE 2

| | Benzene | Toluene | Xylene | C9+ Aromatic | Others |
|---|---|---|---|---|---|
| Ex. 8 | 7.2 | 51.2 | 34.2 | 5.2 | 2.2 |
| Ex. 9 | 5.6 | 37.4 | 46.6 | 8.2 | 2.2 |
| Ex. 10 | 2.0 | 32.6 | 59.2 | 4.6 | 1.6 |
| Ex. 11 | 2.6 | 32.1 | 60.2 | 2.6 | 2.5 |
| Ex. 12 | 9.0 | 39.0 | 42.1 | 6.8 | 3.1 |

### EXAMPLE 13

10 g of byproducts of aromatic carboxylic acid preparation processes composed mainly of BZA (21.4%), TAD (41.4%), CBA (8.3%) and IPA (23.2%), 50 g of hexadecane, 1.0 g of a non-supported MoS₂ catalyst, and hydrogen were fed into a batch reactor, and then reacted under conditions of a reaction temperature of 320° C., a reaction pressure of 55 atm and a reaction time of 8 hr, producing aromatic hydrocarbon compounds composed mainly of benzene, toluene and xylene.

Preparation of Non-Supported MoS₂ Catalyst

5 g of ammonium tetrathiomolybdate and 50 g of hexadecane were fed into a batch reactor, after which H₂ (containing 10% H₂S) was added thereto at room temperature and at 30 atm, and the resulting mixture was heated to 330° C. and then allowed to stand at this temperature for 3 hr. Examples of the Mo precursor of the present invention include not only ammonium tetrathiomolybdate but also a variety of Mo precursors, and the present invention is not limited thereto.

### EXAMPLE 14

20 wt % of byproducts of aromatic carboxylic acid alkylester preparation processes composed mainly of MBZ (43.5%) and MT (44.9%) was dissolved in hexadecane, after which 0.1 wt % of dimethyldisulfide was added thereto, thus preparing a reactant, which was then fed at a rate of 0.2 cc/min into a fixed-bed continuous reactor filled with 5 cc of a MoS₂/Al₂O₃ catalyst. As such, the reaction was carried out under conditions of a reaction temperature of 350° C., a reaction pressure of 30 atm and H₂=20 cc/min, producing aromatic hydrocarbon compounds composed mainly of benzene, toluene and xylene.

Preparation and Sulfidation of MoS₂/Al₂O₃ Catalyst

This catalyst was prepared by supporting about 15 wt % of Mo on an alumina support having a diameter of 1 mm. Specifically, the alumina support was impregnated with an aqueous solution of AHM in distilled water, dried at 150° C. for 2 hr, and continuously fired at 500° C. for 2 hr, thus preparing MoO₃/Al₂O₃. The prepared catalyst was sulfided in the same manner as in Example 1, with the exception that the Sulfidation temperature was 350° C.

### EXAMPLE 15

20 wt % of a mixture of byproducts of aromatic carboxylic acid and/or aromatic carboxylic acid alkylester preparation processes composed mainly of MBZ (19.5%), TAD (37.7%), CBA (7.5%), IPA (21.1%), MBZ (1.1%), MT (1.7%), MFB (5.0%) and DMT (0.9%) was mixed with ethylbenzene, after which 0.5 wt % of dimethyldisulfide was added thereto, thus preparing a reactant, which was then fed at a rate of 0.5 cc/min into a fixed-bed continuous reactor filled with 5 cc of a RuS/carbon catalyst. As such, the reaction was carried out under conditions of a reaction temperature of 330° C., a reaction pressure of 30 atm and H₂=60 cc/min, producing aromatic hydrocarbon compounds composed mainly of benzene, toluene, and xylene.

Preparation of Ru/Carbon Catalyst

This catalyst was prepared by supporting about 7 wt % of Ru on a carbon support having a diameter of 1 mm. Specifically, the carbon support was impregnated with an aqueous solution of ruthenium (III) chloride in distilled water, and continuously dried at 150° C. for 6 hr, thus preparing a Ru/carbon catalyst. The Ru precursor is not limited to ruthenium (III) chloride, and a variety of Ru precursors may be used in the present invention.

Sulfidation of Catalyst

5 cc of the catalyst prepared as above was charged into a fixed-bed continuous reactor, heated to 350° C. while allowing H₂ to flow at a rate of 16 cc/min (H₂S 10 mol %) at room temperature and at a reaction pressure of 45 atm, and then pretreated at 350° C. for 3 hr.

### EXAMPLE 16

20 wt % of byproducts of aromatic carboxylic acid alkylester preparation processes composed mainly of MBZ (11.9%), MT (18.7%), MFB (54.8%) and DMT (9.6%) was dissolved in ethylbenzene, after which 0.5 wt % of dimethyldisulfide was added thereto, thus preparing a reactant, which was then fed at a rate of 0.2 cc/min into a fixed-bed continuous reactor filled with 5 cc of a FeS/clay catalyst. As such, the reaction was carried out under conditions of a reaction temperature of 350° C., a reaction pressure of 40 atm and H₂=20 cc/min, producing aromatic hydrocarbon compounds composed mainly of benzene, toluene and xylene.

Preparation and Sulfidation of Fe/Clay Catalyst

This catalyst was prepared by supporting about 10 wt % of Fe on a clay support having a diameter of 1 mm. Specifically, the clay support was impregnated with an aqueous solution of iron (III) nitrate in distilled water, dried at 150° C. for 2 hr, and continuously fired at 500° C. for 2 hr, thus preparing a Fe/clay catalyst, which was then sulfided in the same manner as in Example 15. The clay used was montmorillonite, and the clay and the Fe precursor are not limited thereto.

### EXAMPLE 17

20 wt % of byproducts of aromatic carboxylic acid preparation processes composed mainly of BZA (21.4%), TAD (41.4%), CBA (8.3%) and IPA (23.2%) was mixed with ethylbenzene, after which 0.5 wt % of dimethyldisulfide was added thereto, thus preparing a reactant, which was then fed at a rate of 0.3 cc/min into a fixed-bed continuous reactor filled with 5 cc of a NiS/Al₂O₃ catalyst. As such, the reaction was carried out under conditions of a reaction temperature of 350° C., a reaction pressure of 30 atm and H₂=30 cc/min, producing aromatic hydrocarbon compounds composed mainly of benzene, toluene, and xylene.

Preparation and Sulfidation of Ni/Al₂O₃ Catalyst

This catalyst was prepared by supporting about 10 wt % of Ni on an alumina support having a diameter of 1 mm. Specifically, the alumina support was impregnated with an aqueous solution of NNH in distilled water, dried at 150° C. for 2 hr, and continuously fired at 500° C. for 2 hr, thus preparing a Ni/Al₂O₃ catalyst, which was then sulfided in the same manner as in Example 15. As such, the Ni precursor is not limited to NNH, and a variety of Ni precursors may be used in the present invention.

### EXAMPLE 18

20 wt % of byproducts of aromatic carboxylic acid alkylester preparation processes composed mainly of MBZ (11.9%), MT (18.7%), MFB (54.8%) and DMT (9.6%) was dissolved in ethylbenzene, after which 0.3 wt % of dimethyldisulfide was added thereto, thus preparing a reactant, which was then fed at a rate of 0.3 cc/min into a fixed-bed continuous reactor filled with 5 cc of a ReS/zeolite catalyst. As such, the reaction was carried out under conditions of a reaction temperature of 320° C., a reaction pressure of 20 atm and H₂=30 cc/min, producing aromatic hydrocarbon compounds composed mainly of benzene, toluene, and xylene.

Preparation and Sulfidation of Re/Zeolite Catalyst

This catalyst was prepared by supporting about 5 wt % of Re on a zeolite support having a diameter of 1 mm. Specifically, the zeolite support was impregnated with an aqueous solution of ammonium perrhenate in distilled water, dried at 150° C. for 2 hr, and continuously fired at 500° C. for 2 hr, thus preparing a Re/zeolite catalyst, which was then sulfided in the same manner as in Example 15. As such, the zeolite used was ZSM-5, and the zeolite and the Re precursor are not limited thereto.

### EXAMPLE 19

20 wt % of byproducts of aromatic carboxylic acid preparation processes composed mainly of BZA (21.4%), TAD (41.4%), CBA (8.3%) and IPA (23.2%) was mixed with ethylbenzene, after which 0.5 wt % of dimethyldisulfide was added thereto, thus preparing a reactant, which was then fed at a rate of 0.2 cc/min into a fixed-bed continuous reactor filled with 5 cc of a CoS/ZrO₂ catalyst. As such, the reaction was carried out under conditions of a reaction temperature of 350° C., a reaction pressure of 30 atm and H₂=20 cc/min, producing aromatic hydrocarbon compounds composed mainly of benzene, toluene, and xylene.

Preparation and Sulfidation of Co/ZrO₂ Catalyst

This catalyst was prepared by supporting about 10 wt % of Co on a zirconia support having a diameter of 1 mm. Specifically, the zirconia support was impregnated with an aqueous solution of CNH in distilled water, dried at 150° C. for 2 hr, and continuously fired at 500° C. for 2 hr, thus preparing a Co/ZrO₂ catalyst, which was then sulfided in the same manner as in Example 15. As such, the Co precursor is not limited to CNH, and a variety of Co precursors may be used in the present invention.

### EXAMPLE 20

20 wt % of a mixture of byproducts of aromatic carboxylic acid and/or aromatic carboxylic acid alkylester preparation processes composed mainly of MBZ (19.5%), TAD (37.7%), CBA (7.5%), IPA (21.1%), MBZ (1.1%), MT (1.7%), MFB (5.0%) and DMT (0.9%) was mixed with ethylbenzene, after which 0.5 wt % of dimethyldisulfide was added thereto, thus preparing a reactant, which was then fed at a rate of 0.3 cc/min into a fixed-bed continuous reactor filled with 5 cc of a WS₂/TiO₂ catalyst. As such, the reaction was carried out under conditions of a reaction temperature of 340° C., a reaction pressure of 30 atm and H₂=30 cc/min, producing aromatic hydrocarbon compounds composed mainly of benzene, toluene, and xylene.

Preparation and Sulfidation of WS₂/TiO₂ Catalyst

This catalyst was prepared by supporting about 15 wt % of W on a titania support having a diameter of 1 mm. Specifically, the titania support was impregnated with an aqueous solution of AMT in distilled water, dried at 150° C. for 2 hr, and continuously fired at 500° C. for 2 hr, thus preparing WoO₃/TiO₂, which was then sulfided in the same manner as in Example 14.

As such, the W precursor is not limited to AMT, and a variety of W precursors may be used in the present invention.

### EXAMPLE 21

20 wt % of byproducts of aromatic carboxylic acid alkylester preparation processes composed mainly of MBZ (11.9%), MT (18.7%), MFB (54.8%) and DMT (9.6%) was dissolved in ethylbenzene, after which 0.5 wt % of dimethyldisulfide was added thereto, thus preparing a reactant, which was then fed at a rate of 0.3 cc/min into a fixed-bed continuous reactor filled with 5 cc of a PtS/SiO₂ catalyst. As such, the reaction was carried out under conditions of a reaction temperature of 340° C., a reaction pressure of 30 atm and H₂=30 cc/min, producing aromatic hydrocarbon compounds composed mainly of benzene, toluene, and xylene.

Preparation of PtS/SiO₂ Catalyst

This catalyst was prepared by supporting about 5 wt % of Pt on a silica support having a diameter of 1 mm. Specifically, the silica support was impregnated with an aqueous solution of chloroplatinic acid in distilled water, dried at 150° C. for 2 hr, and continuously fired at 500° C. for 2 hr, thus preparing Pt/SiO₂, which was then sulfided in the same manner as in Example 15.

As such, the Pt precursor is not limited to chloroplatinic acid, and a variety of Pt precursors may be used in the present invention.

### EXAMPLE 22

20 wt % of a mixture of byproducts of aromatic carboxylic acid and/or aromatic carboxylic acid alkylester preparation processes composed mainly of MBZ (19.5%), TAD (37.7%), CBA (7.5%), IPA (21.1%), MBZ (1.1%), MT (1.7%), MFB (5.0%) and DMT (0.9%) was dissolved in ethylbenzene, after which 0.3 wt % of dimethyldisulfide was added thereto, thus preparing a reactant, which was then fed at a rate of 0.3 cc/min into a fixed-bed continuous reactor filled with 5 cc of a PdS/Al₂O₃ catalyst. As such, the reaction was carried out under conditions of a reaction temperature of 350° C., a reaction pressure of 30 atm and H₂=30 cc/min, producing aromatic hydrocarbon compounds composed mainly of benzene, toluene and xylene.

Preparation of PdS/Al₂O₃ Catalyst

This catalyst was prepared by supporting about 10 wt % of Pd on an alumina support having a diameter of 1 mm. Specifically, the alumina support was impregnated with an aqueous solution of palladium chloride in distilled water, dried at 150° C. for 2 hr, and continuously fired at 500° C. for 2 hr, thus preparing Pd/Al₂O₃, which was then sulfided in the same manner as in Example 15.

As such, the Pd precursor is not limited to palladium chloride, and a variety of Pd precursors may be used in the present invention.

### EXAMPLE 23

20 wt % of byproducts of aromatic carboxylic acid alkylester preparation processes composed mainly of MBZ (11.9%), MT (18.7%), MFB (54.8%) and DMT (9.6%) was dissolved in ethylbenzene, after which 0.1 wt % of dimethyldisulfide was added thereto, thus preparing a reactant, which was then fed at a rate of 0.3 cc/min into a fixed-bed continuous reactor filled with 5 cc of a RhS/ZrO₂ catalyst. As such, the reaction was carried out under conditions of a reaction temperature of 300° C., a reaction pressure of 20 atm and H₂=30 cc/min, producing aromatic hydrocarbon compounds composed mainly of benzene, toluene and xylene.

Preparation of RhS/ZrO₂ Catalyst

This catalyst was prepared by supporting about 10 wt % of Rh on a zirconia support having a diameter of 1 mm. Specifically, the zirconia support was impregnated with an aqueous solution of rhodium (III) chloride in distilled water, dried at 150° C. for 2 hr, and continuously fired at 500° C. for 2 hr, thus preparing Rh/ZrO₂, which was then sulfided in the same manner as in Example 15.

As such, the Rh precursor is not limited to rhodium chloride, and a variety of Rh precursors may be used in the present invention.

### EXAMPLE 24

20 wt % of byproducts of aromatic carboxylic acid alkylester preparation processes composed mainly of MBZ (43.5%) and MT (44.9%) was dissolved in ethylbenzene, after which 0.1 wt % of dimethyldisulfide was added thereto, thus preparing a reactant, which was then fed at a rate of 0.3 cc/min into a fixed-bed continuous reactor filled with 5 cc of a CrS/ZrO₂ catalyst. As such, the reaction was carried out under conditions of a reaction temperature of 350° C., a reaction pressure of 50 atm and H₂=30 cc/min, producing aromatic hydrocarbon compounds composed mainly of benzene, toluene and xylene.

Preparation of CrS/ZrO₂ Catalyst

This catalyst was prepared by supporting about 10 wt % of Cr on a zirconia support having a diameter of 1 mm. Specifically, the zirconia support was impregnated with an aqueous solution of chromium (III) nitrate in distilled water, dried at 150° C. for 2 hr, and continuously fired at 500° C. for 2 hr, thus preparing Cr/ZrO₂, which was then sulfided in the same manner as in Example 15. As such, the Cr precursor is not limited to chromium nitrate, and a variety of Cr precursors may be used in the present invention.

The compositions of the liquid reaction products of Examples 13 to 24 and the amounts (unit: %) thereof are given in Table 3 below (hexadecane or ethylbenzene used as the mixing solvent is not shown).

TABLE 3

| | Benzene | Toluene | Xylene | C9+ Aromatic | Others |
|---|---|---|---|---|---|
| Ex. 13 | 6.9 | 44.5 | 38.6 | 7.0 | 3.0 |
| Ex. 14 | 23.7 | 48.5 | 26.7 | 0.9 | 0.2 |
| Ex. 15 | 5.4 | 37.9 | 47.6 | 6.6 | 2.5 |
| Ex. 16 | 3.6 | 38.6 | 52.8 | 3.4 | 1.6 |
| Ex. 17 | 6.4 | 35.4 | 50.2 | 5.7 | 2.3 |
| Ex. 18 | 2.1 | 34.6 | 57.4 | 3.7 | 2.2 |
| Ex. 19 | 6.8 | 39.4 | 44.3 | 6.5 | 3.0 |
| Ex. 20 | 5.8 | 38.0 | 46.0 | 6.4 | 3.8 |
| Ex. 21 | 3.4 | 33.0 | 58.8 | 2.8 | 2.0 |
| Ex. 22 | 6.3 | 39.3 | 46.2 | 5.6 | 2.6 |
| Ex. 23 | 2.9 | 32.6 | 59.0 | 3.2 | 2.3 |
| Ex. 24 | 23.3 | 47.1 | 26.2 | 1.2 | 2.2 |

### EXAMPLE 25

20 wt % of byproducts of aromatic carboxylic acid preparation processes composed mainly of BZA (21.4%), TAD (41.4%), CBA (8.3) and IPA (23.2%) was mixed with ethylbenzene, thus preparing a reactant, which was then fed at a rate of 0.5 cc/min into a fixed-bed continuous reactor filled with 5 cc of a MoOₓ/ZrO₂ catalyst. As such, the reaction was carried out under conditions of a reaction temperature of 350° C., a hydrogen pressure of 40 atm and H₂=50 cc/min, producing aromatic hydrocarbon compounds composed mainly of benzene, toluene and xylene.

Preparation and Pretreatment of MoOₓ/ZrO₂ Catalyst

This catalyst was prepared by supporting about 8 wt % of Mo on a zirconia support having a diameter of 1 mm. Specifically, the zirconia support was impregnated with an aqueous solution of AHM in distilled water, dried at 150° C. for 2 hr, and continuously fired at 500° C. for 2 hr, thus preparing MoO₃/ZrO₂. 5 cc of the catalyst prepared as above was charged into a fixed-bed continuous reactor, heated to 400° C. while allowing H₂ to flow at a rate of 100 cc/min at room temperature and at a reaction pressure of 40 atm, and then pretreated at 400° C. for 3 hr, thereby obtaining MoOₓ/ZrO₂.

### EXAMPLE 26

20 wt % of byproducts of aromatic carboxylic acid preparation processes composed mainly of BZA (21.4%), TAD (41.4%), CBA (8.3) and IPA (23.2%) was mixed with ethylbenzene, thus preparing a reactant, which was then reacted in the same manner as in Example 25.

Preparation and Pretreatment of WOₓ/TiO₂ Catalyst

This catalyst was prepared by supporting about 8 wt % of W on a titania support having a diameter of 1 mm. Specifically, the titania support was impregnated with an aqueous solution of AMT in distilled water, dried at 150° C. for 2 hr, and continuously fired at 500° C. for 2 hr, thus preparing WO₃/TiO₂. 5 cc of the catalyst prepared as above was charged into a fixed-bed continuous reactor, heated to 400° C. while allowing H₂ to flow at a rate of 100 cc/min at room temperature and at a reaction pressure of 40 atm, and then pretreated at 400° C. for 3 hr, thereby obtaining WOₓ/TiO₂.

The compositions of the liquid reaction products of Examples 25 and 26 and the amounts (unit: %) thereof are given in Table 4 below (ethylbenzene used as the mixing solvent is not shown).

TABLE 4

| | Benzene | Toluene | Xylene | C9+ Aromatic | Others |
|---|---|---|---|---|---|
| Ex. 25 | 6.0 | 35.2 | 47.5 | 8.8 | 2.5 |
| Ex. 26 | 6.9 | 34.7 | 48.2 | 7.6 | 2.6 |

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method of producing aromatic hydrocarbons, comprising:
a) preparing byproducts of either or both of aromatic carboxylic acid preparation processes and aromatic carboxylic acid alkylester preparation processes; and
b) converting the byproducts into the aromatic hydrocarbons using a hydroprocessing catalyst, thus obtaining a product including the aromatic hydrocarbons.

2. The method of claim 1, further comprising c) separating the product obtained in b) into a liquid aromatic hydrocarbons, gas components and water.

3. The method of claim 2, further comprising d) separating the liquid aromatic hydrocarbons obtained in c) into benzene, toluene, xylene, and hydrocarbon compounds having 9 or more carbons.

4. The method of claim 2, further comprising e) recycling a part of the liquid aromatic hydrocarbons, the gas components, or hydrogen of the gas components from which impurities have been removed, as obtained in c), into a) so as to be mixed with the byproducts.

5. The method of claim 3, further comprising f) recycling a part of the aromatic hydrocarbons obtained in d) into a) so as to be mixed with the byproducts.

6. The method of claim 1, wherein the aromatic carboxylic acid is terephthalic acid, isophthalic acid, phthalic acid and anhydrides thereof, or benzoic acid, and the aromatic carboxylic acid alkylester is dimethyl terephthalate, dimethyl isophthalate or dimethyl phthalate.

7. The method of claim 1, wherein the aromatic carboxylic acid is terephthalic acid, and the aromatic carboxylic acid alkylester is dimethyl terephthalate.

8. The method of claim 1, wherein the byproducts include aromatic compounds in which a benzene ring has a substituent that contain hydrocarbons or oxygen.

9. The method of claim 8, wherein the substituent is an alkyl group, an aldehyde group, a carboxyl group, an ester group, a hydroxyl group, or an alkoxy group.

10. The method of claim 1, wherein b) is performed under conditions of a reaction temperature of 150˜500° C. and a reaction pressure of 1˜300 kgf/cm².

11. The method of claim 1, wherein the hydroprocessing catalyst is provided in a non-supported form or in a form of being supported on an inorganic oxide or carbon support.

12. The method of claim 11, wherein the support is one or more selected from the group consisting of silica, alumina, silica-alumina, zirconia, titania, aluminum phosphate, niobia, clay, zeolite and carbon.

13. The method of claim 1, wherein the hydroprocessing catalyst includes one or more metals selected from the group consisting of metals of Groups 6, 7, 8, 9, 10 and 11 on a periodic table, or sulfide, phosphide or oxide thereof.

14. The method of claim 13, wherein the metal is one or more selected from the group consisting of Cr, Mo, W, Re, Ru, Co, Rh, Ni, Pd, Pt, Cu, and Fe.

15. The method of claim 13, wherein the metal is one or more selected from the group consisting of Mo, W, Fe, Ru, Co, Ni, and Cu.
